(19) United States
(12) Patent Application Publication
(10) Pub. No.: US 2023/0233488 A1
(43) Pub. Date: Jul. 27, 2023

(54) NOVEL USE OF A MODULATOR OF GLUCOSYLCERAMIDE DEGRADATION FOR VIRAL INFECTIONS

(71) Applicant: SPEDDING RESEARCH SOLUTIONS, SAS, LE VESINET (FR)

(72) Inventors: Michaël SPEDDING, LE VESINET (FR); Rushika Michelle PERERA, FORT COLLINS, CO (US); Laura Athelle ST. CLAIR, ARVADA, CO (US)

(73) Assignee: SPEDDING RESEARCH SOLUTIONS, SAS, LE VESINET (FR)

(21) Appl. No.: 18/001,762

(22) PCT Filed: Jun. 21, 2021
(86) PCT No.: PCT/EP2021/066314

§ 371 Date: Dec. 14, 2022

Related U.S. Application Data
(60) Provisional application No. 63/039,767, filed on Jun. 16, 2020.

(51) Int. Cl. A61K 31/136; A61K 45/06; A61P 31/14

(52) U.S. Cl. CPC A61K 31/136; A61K 45/06; A61P 31/14

(57) ABSTRACT

A modulator of glucosylceramide degradation and pharmaceutical compositions containing the same. Also, the use of the modulator of glucosylceramide degradation and pharmaceutical compositions containing the same in the treatment or the prevention of viral infections and disorders associated to the viral infections, such as Zika infection, dengue infection, influenza infection and coronavirus infection.

### FIELD

The present invention relates to modulator of glucosylceramide degradation, particularly ambroxol, to pharmaceutical compositions containing same and to the use of same in the treatment and/or in the prevention of viral infections and disorders associated to the viral infections, such as Zika infection, dengue infection, influenza infection and coronavirus infection.

### BACKGROUND

Many coronaviruses, first discovered in domestic poultry in the 1930s, cause respiratory, gastrointestinal, liver and neurological diseases in animals. Only seven coronaviruses are known to cause disease in humans. Four of the seven coronaviruses most often cause cold symptoms. Coronaviruses 229E and OC43 cause the common cold; serotypes NL63 and HUK1 have also been associated with colds. Rarely, serious lower respiratory infections, including pneumonia, can occur, mainly in infants, the elderly, and immunocompromised individuals.

Three of the seven coronaviruses cause much more serious and sometimes fatal respiratory infections in humans and have caused major epidemics of deadly pneumonia in the 21st century:
SARS-CoV-2 or “COVID-19” (also mentioned as “SARS-COV 2” or “SARS-COV-2” or “SARS-CoV 2”) is a new coronavirus identified as the cause of 2019 coronavirus disease (COVID-19) which started in Wuhan, China in late 2019 and has spread around the world.
MERS-CoV (also mentioned as “MERS-COV”) was identified in 2012 as the cause of the Middle East respiratory syndrome (MERS [Middle East respiratory syndrome]).
SARS-CoV or “SARS-CoV-1 (also mentioned as “SARS-COV” or “SARS-CoV 1” or “SARS-COV 1” or “SARS-COV-1”) was identified in 2002 as the cause of an epidemic of severe acute respiratory syndrome (SARS).

Influenza infection, commonly known as “the flu”, is an infectious disease caused by an influenza virus. Symptoms can be mild to severe. The most common symptoms include: high fever, runny nose, sore throat, muscle and joint pain, headache, coughing, and feeling tired. These symptoms typically begin two days after exposure to the virus and most last less than a week. The cough, however, may last for more than two weeks. In children, there may be diarrhea and vomiting, but these are not common in adults. Diarrhea and vomiting occur more commonly in gastroenteritis, which is an unrelated disease and sometimes inaccurately referred to as “stomach flu” or the “24-hour flu”. Complications of influenza may include viral pneumonia, secondary bacterial pneumonia, sinus infections, and worsening of previous health problems such as asthma or heart failure. A vaccine made for one year may not be useful in the following year, since the virus evolves rapidly. Antiviral medications such as the neuraminidase inhibitor or oseltamivir, among others, have been used to treat influenza. The benefit of antiviral medications in those who are otherwise healthy do not appear to be greater than their risks.

Zika infection, also known as Zika fever or Zika virus disease, often causes no or only mild symptoms, similar to a very mild form of dengue fever. While there is no specific treatment, paracetamol and rest may help with the symptoms. As of April 2019, no vaccines have been approved for clinical use. Zika can spread from a pregnant woman to her baby. This can result in microcephaly, severe brain malformations, and other birth defects. Zika infections in adults may result rarely in Guillain-Barré syndrome.

Dengue infection, also known as dengue fever, is a mosquito-borne tropical disease caused by the dengue virus which is a single positive-stranded RNA virus of the family Flaviviridae; genus Flavivirus. La dengue est due à un arbovirus (virus transmis par les insectes), appartenant à la famille des Flaviviridae, du genre flavivirus, comme le virus West Nile et de la fièvre jaune. Il est transmis à l'homme par les moustiques du genre Aedes lors d'un repas sanguin. Four serotypes of the virus have been found, all of which can cause the full spectrum of disease DENV-1, DENV-2, DENV-3, and DENV-4. Immunity acquired in response to infection with one of the serotypes confers protective immunity against the infecting serotype but not against the other serotypes. As a result, an individual is likely to be infected with all four serotypes of dengue during his or her lifetime. Subsequent infections with other serotypes increase the risk of developing severe dengue fever. An effective dengue vaccine should therefore be able to confer protective immunity against all serotypes. Symptoms typically begin three to fourteen days after infection. These may include a high fever, headache, vomiting, muscle and joint pains, and a characteristic skin rash. Recovery generally takes two to seven days. In a small proportion of cases, the disease develops into severe dengue, also known as dengue hemorrhagic fever, resulting in bleeding, low levels of blood platelets and blood plasma leakage, or into dengue shock syndrome, where dangerously low blood pressure occurs (Kristin et al. 2018). The authors consider that there is no specific treatment and that paracetamol and rest may help with the symptoms; these symptomatic treatments (co-administered in hospitals in Indonesia) are listed by these authors, who also indicate that there are 390 million infections/year worldwide, with 96 million cases.

Today, there is no antiviral drug or no other specific treatment, other than certain vaccines, such as those developed for COVID-19, to prevent and/ or treat viral infections, and in particular to prevent and/or treat infections caused by enveloped viruses, and in particular Zika infection, dengue infection, influenza infection and coronavirus infection.

Consequently, it appears necessary to have new compositions capable of blocking virus multiplication, in order to prevent and/or treat viral infections, in particular Zika infection, dengue infection, influenza infection and coronavirus infection, while reducing the risk of side effects, when administered to the patient.

### SUMMARY

The present invention relates to a modulator of glucosylceramide degradation or a pharmaceutical acceptable salt thereof, as active ingredient, in an effective amount for treating or preventing viral infections and disorders associated to the viral infections.

The present invention also relates to a modulator of glucosylceramide degradation or a pharmaceutical acceptable salt thereof, as active ingredient, in an effective amount for its use for treating or preventing viral infections and disorders associated to the viral infections.

Surprisingly, the inventors have shown that the modulator of glucosylceramide degradation, and in particular ambroxol, inhibits the replication of enveloped viruses, and in particular inhibits the replication of the SARS-CoV-2 virus and of the dengue virus. In particularly, the inventors have shown that sphingolipids (GSLs) are essential in the infection cycle of enveloped human RNA viruses, which are critically dependent on host cell lipid synthesis.

Furthermore, the inventors have shown that the modulator of glucosylceramide degradation is able to inhibit viral multiplication by inhibiting glucosylceramide degradation. The inventors have also shown that the modulator of glucosylceramide degradation has major effects in viral infections due to SARS-CoV-2, by blocking the access of the virus to the lungs, by blocking the access of the virus to ACE2/TMPRSS2 and lipid rafts and thus inhibiting the internalization of the virus. The inventors have also shown that the modulator of glucosylceramide degradation is able to protect the lungs and the cardiac function of the patient suffering from viral infection due to SARS-CoV-2, but also to protect the patient suffering from viral infection due to SARS-CoV-2 from muscle loss, and recovery from intensive care. The inventors have also shown that the modulator of glucosylceramide degradation is able to modulate the mitochondrial function.

### BRIEF DESCRIPTION OF THE FIGURES

FIG. 1 concerns the antiviral activity of Ambroxol against SARS-CoV-2 in A549-ACE2 cells. In 1A, viral load (genome equivalents) in supernatants were measured at 48 h [symbol] , left Y axis), and cell viability under increasing concentrations of the antiviral compound is shown [symbol] right Y axis). In these experiments the multiplicity of infection (MOI) was 0.1. In 1B, same data with viral load reported as a ratio of the no treatment condition (DMSO). 1C shows the supernatant viral load in a further experiment over a tighter concentration range of ambroxol, with a 50% effective concentration of 22 μM.

FIG. 2 shows the role of glucosylceramide hydrolysis on the control of DENV2 lifecycle as measured according to example 2. In 2A, after inhibition of GBA1, the amount of infectious virus released in the supernatant was measured at 24 h and compared to a non-inhibitory control (IRR). In 2B-C, the antiviral activity of ambroxol against DENV2 infection of Huh7 cells was assessed by measuring infectious virus released into supernatants (black curve left Y axis), along with a concurrent cell viability assessment under increasing concentrations (grey curve right Y axis). In 2B, cells were treated with virus at a low multiplicity of infection (MOI=0.1), and 2C cells were infected with virus at a high multiplicity of infection (MOI=5). In 2D-G, Huh7 cells were treated with siRNAs that inhibit GBA1 and GBA2, and infected with DENV2 at both low and high MOI (low MOI=0.1, 2D-E, high MOI=5, 2F-G). Following 24 h of infection, viral output was measured via infectious virus release in supernatants (2D,F) or via copy number of DENV2 mRNA from infected cells (genome equivalents, 2E,G). In 2A, D-G infectious virus release or viral copy number is compared to a non-inhibitory control (IRR).

The statistical significance of the differences observed for the different conditions was evaluated by a Student t-test * p<0.05; ** p<0.1; ***p<0.005; ****p<0.001.

### DETAILED DESCRIPTION

As used herein, the term “modulator” refers to a composition that modulates one or more physiological or biochemical events associated with viral infection and disorders associated to the viral infection. As used herein, a “modulator of glucosylceramide degradation” is a compound which is able to modulate the activity of beta-glucocerebrosidase 1 (GBA1) or which is able to modulate the activity beta-glucocerebrosidase 2 (GBA2) or both.

As used herein, the term “modulate” or “modulating” means changing or altering, and embraces both upmodulating or upregulating and downmodulating or down regulating. In one embodiment, the modulator of glucosylceramide degradation according to the invention is able to change or alter the activity of beta-glucocerebrosidase 1 (GBA1) or the activity of beta-glucocerebrosidase 2 (GBA2) or both. Advantageously, the modulator of glucosylceramide degradation according to the invention is able to upregulate the activity of beta-glucocerebrosidase 1 (GBA1) and downregulate the activity of beta-glucocerebrosidase 2 (GBA2).

Throughout this application, it is contemplated that the term “compound” or “compounds” refers to the compounds discussed herein and includes precursors and derivatives of the compounds, including acyl-protected derivatives, and pharmaceutically acceptable salts of the compounds, precursors, and derivatives.

In an advantageous embodiment, the modulator of glucosylceramide degradation used according to the invention is used as unique active ingredient for treating or preventing viral infections and disorders associated to the viral infections.

In an advantageous embodiment, the modulator of glucosylceramide degradation according to the invention promotes the activity of beta-glucocerebrosidase 1 (GBA or GBA1) or inhibit the activity of beta-glucocerebrosidase 2 (GBA2). Said enzymes catalyze the hydrolytic cleavage of the beta-glucosidic linkage of the glucosylceramide (GlcCer). Alternative names for GBA include GBA1, acid beta-glucosidase, beta-GC, D-glucosyl-N-acylsphingosine glucohydrolase or lysosomal beta-glucocerebrosidase. GBA2 is also known as non-lysosomal beta-glucocerebrosidase All these term are equivalent. In an advantageous embodiment, the modulator of glucosylceramide degradation according to the invention may promote the activity of beta-glucocerebrosidase 1 (GBA or GBA1) and inhibit the activity of beta-glucocerebrosidase 2 (GBA2). In an advantageous embodiment, the modulator of glucosylceramide degradation according to the invention may inhibit or may act as an enzyme chaperone, binding to the enzyme to increase enzyme transport into lysosomes and thereby increasing lysosomal glucosidase activity specifically.

As used herein, the term “inhibit” refers to a decrease in an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10%, advantageously a 20%, advantageously a 30%, advantageously a 40%, advantageously a 50%, advantageously a 60%, advantageously a 70%, advantageously a 80%, advantageously a 90%, advantageously a 100%, or any amount of reduction in between as compared to native or control levels. Advantageously, the modulator of glucosylceramide degradation can reduce the activity of GBA1 or GBA2 or GBA1 and GBA2 by 10%, advantageously by 20%, advantageously by 30%, advantageously by 40%, advantageously by 50%, advantageously by 60%, advantageously by 70%, advantageously by 80%, advantageously by 90%, advantageously by 100%. Advantageously, the modulator of glucosylceramide degradation is able to inhibit the cleavage of glucose from GlcCer. In some embodiment one or more of the compounds according to the invention may inhibit a GCase within a specific cellular compartment, such as the endoplasmic reticulum or Golgi apparatus, but may dissociate and no longer inhibit a GCase within another cellular compartment, for example a lysosomal compartment.

In an advantageous embodiment, the modulator of glucosylceramide degradation is ambroxol or a pharmaceutically acceptable salt thereof. Ambroxol, also named [trans-4-(2-Amino-3,5-dibromobenzylamino) cyclohexanol hydrochloride] is a modulator of glucosylceramidase enzymes, without inhibition of glucosylceramide synthase, GCS, or UGCG (UDP-Glucose Ceramide Glucosyltransferase). Ambroxol is particularly used as expectorant or as anti-inflammatory and/or anti-oxidant agent.

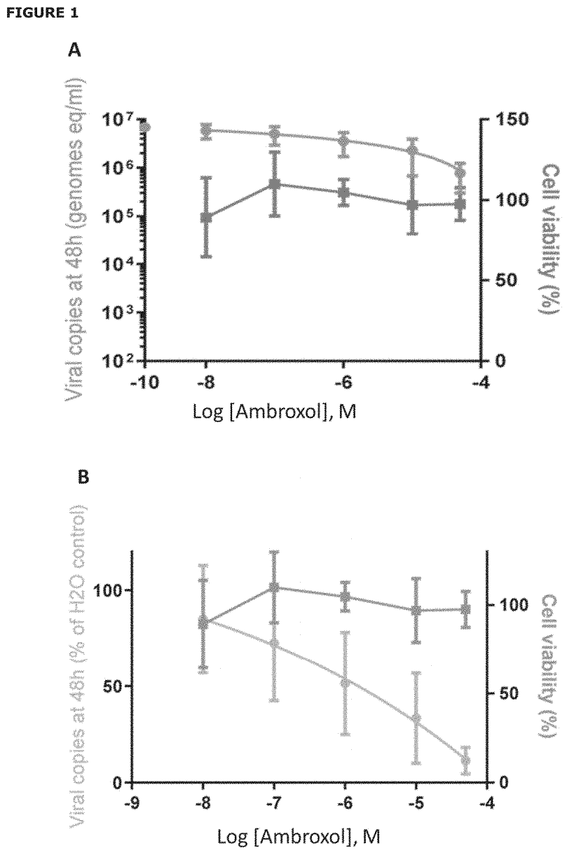

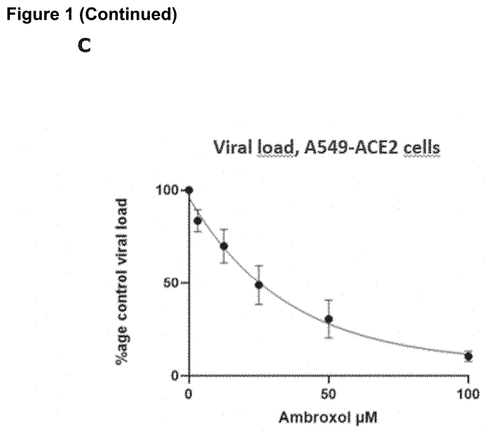

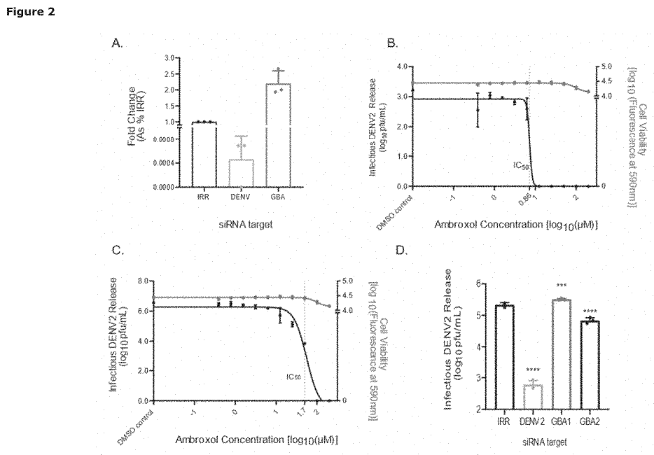

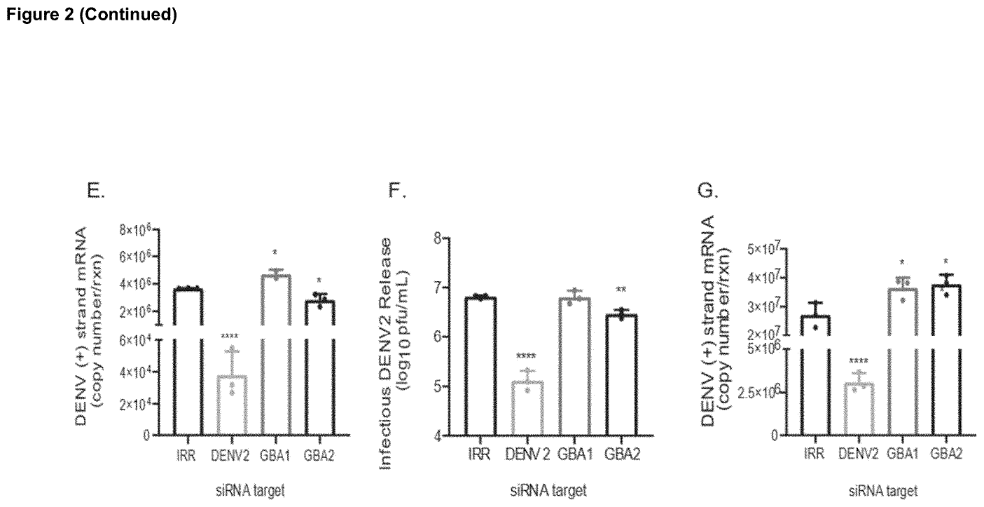

Surprisingly, the inventors have shown that glucosylceramide is a critical node in SARS-CoV-2 infection of host cells, and of Dengue infection of host cells by using siRNA technology. They have also shown that the ambroxol inhibits the replication of enveloped viruses, and in particular inhibits the replication of the SARS-CoV-2 virus (FIG. 1) and dengue virus, being 10-fold more potent against Dengue virus (FIG. 2). Ambroxol is a subtype selective modulator of glucocerebrosidase (GCase), the critical enzyme for production of many glycosphingolipids (GSLs), and which preserves GM1 levels. GM1 is a critical neurotrophin with human-specific sialic acid residues, which is also the target for influenza. Surprisingly, the inventors have shown that the same enzymes are used by envelope viruses to pervert host metabolism. More particularly, the inventors have shown that sphingolipids (GSLs) are essential in the infection cycle of enveloped human RNA viruses, which are critically dependent on host cell lipid synthesis. There is previous evidence showing that inhibiting glucosylceramide synthase (otherwise known as UGCG, UDP-Glucose Ceramide Glucosyltransferase) may be effective in inhibiting viral replication which is reviewed by (Vitner et al. 2021). However, inhibitors of GCS may exacerbate some neurological disorders such as amyotrophic lateral sclerosis (ALS); (Henriques et al. 2015; Henriques, Croixmarie, et al. 2017; Henriques, Huebecker, et al. 2017; Dodge et al. 2015), whereas ambroxol is beneficial in these conditions (Bouscary et al. 2020; 2019).

Furthermore, the inventors have shown that ambroxol is able to inhibit viral multiplication by inhibiting glucosylceramide degradation. Indeed, the inventors have shown that ambroxol has a concentration-dependent effect reducing SARS-CoV-2 viral load in A549 cells transfected with angiotensin converting enzyme 2 (ACE2). Advantageously, ambroxol is able to reduce SARS-CoV-2 viral load in A549 cells transfected with angiotensin converting enzyme 2, when ambroxol is used at a concentration of 10⁻⁸ to 5.10⁻⁵ M. In a further series of experiments ambroxol was active with IC50s between 11 μM and 22 μM using an MOI (multiplicity of infection) of 0.1 with MOI being the number of viral particles that can infect each cell in a given experiment; these concentrations are the same concentrations as ambroxol increases the formation of new neuromuscular junctions from spinal explants maintained on myoblasts in culture, an effects attributed to GCase activity by (Bouscary et al. 2019; 2020), when proposing the use of ambroxol for ALS. Ambroxol was 10 fold more potent against Dengue virus than against SARS-CoV-2. with an IC50 for ambroxol of 0.86 μM at MOI 0.1.

The inventors have also shown that ambroxol is able to block the access of the virus to the lungs. Indeed, the spike protein of influenza targets sialic acid residues on target cells such as GM1, but also sialic acids plentiful in mucus. The receptor for influenza viruses and for human coronavirus OC43 is N-Acetylneuraminic acid (Neu5Ac). Thus influenza virus must navigate against ‘decoy’ sialic acids in the 10 μm mucus layer, which propel the virus to the pharynx and inactivation in the stomach before it can penetrate deep into the lung. As SARS-CoV-2 has lower affinity for sialic acids than influenza virus, but preferentially binds angiotensin converting enzyme 2 (ACE2) it can penetrate deeper into the lungs, but increasing mucus flow will help reduce infection and clear the lungs.

The inventors have also shown that ambroxol is able to block the access of the virus to ACE2/TMPRSS2 and lipid rafts and thus inhibiting the internalization of the virus. In the nose and lungs (Ziegler et al. 2020) have found that goblet secretory cells, which produce mucus, express RNAs for both of the cellular targets for SARS-CoV-2, ACE2 and TMPRSS2. In the lungs, they found the RNAs for these proteins mainly in type II pneumocytes. These cells line the alveoli of the lungs and are responsible for keeping them open. Furthermore, (Ziegler et al. 2020) reported that ACE2 and TMPRSS2 are upregulated by interferon-stimulated genes, meaning that there is a unique targeting system of SARS-CoV-2 delivering interferon-suppressing genes directly to interferon-producing cells. Thus, the cells producing mucus are critical sites for the actions of SARS-CoV-2, indicating potential direct antagonism between ambroxol and SARS-CoV-2. Ambroxol is a potent mucolytic agent acting on type II pneumocytes. Type II pneumocytes are a primary target of SARS-CoV-2. Thus, the main sites of action of SARS-CoV-2 and ambroxol converge. Once the SARS-CoV-2 virus arrives at its target, it binds to the ACE2 receptor which is localised in lipid rafts. Lipid rafts are maintained by GSLs and cholesterol, essential to viral entry, thus agents acting on GBA1 and GBA2. Ambroxol will directly affect lipid raft stability, by modulating GBA1 and GBA2.

The inventors have also shown that ambroxol is able to protect the lungs, by inhibiting glucosylceramidase and in particular GBA2. Indeed, patients suffering from SARS-CoV-2 infection have severe acute respiratory syndrome, with evident lung dysfunction. Ceramide is upregulated and deleterious in cystic fibrosis. Ceramide and glucosylceramide play opposing roles in pneumonia, lung inflammation, fibrosis and in cystic fibrosis. Ceramide is increased and is proinflammatory. In contrast glucosylceramide and lactosylceramide are down-regulated on the apical surface of the bronchial and tracheal cells in cystic fibrosis. Glucosylceramide is considered be a critical defense of the lungs in cystic fibrosis, particularly as aerosol administration was protective in models of the disease or following pneumonia induced by pseudomonas aeruginosa. By inhibiting glucosylceramidase and in particular GBA2, which are particularly involved in SARS-CoV-2 infection, ambroxol is able to protect the lungs.

The inventors have also shown that ambroxol protects the cardiac function of patient suffering from viral infections. Many patients may die of thrombosis, stroke, viral myocarditis, arrhythmias and loss of excitation-contraction coupling. This may be due to deranged lipid metabolism caused by viral infection. The protein from Orf-9b (present in both SARS-CoV-1 and SARS-CoV-2) directly targets mitochondria, metabolism, and limits host interferon responses. Ambroxol by targeting lipid metabolism will have an effect on acylcarnitine metabolism and heart function. Ambroxol has effects on mitochondria function and changes lipid metabolism, directly influencing acylcarnitine levels and subsequently lysophospholipids such as lysophosphatidylcholine (LPC) levels. LPC is produced in endothelial cells and myocytes in response to thrombin, involved in activation of blood coagulation. Acylcarnitines are the carriers of lipids into the mitochondria for oxidation, but which, in the presence of ischemia, heart failure, may accumulate in the heart and also cause arrhythmias, cellular uncoupling, and interfere with myocardial excitation-contraction coupling, by activation of calcium channels and intracellular calcium. Thus, ambroxol will protect patients from the sudden cardiac death associated with SARS-CoV-2 infection (also named “COVID-19”).

The inventors have also shown that ambroxol protects patient suffering from muscle loss caused by viral infections, and recovery from intensive care. Indeed, a key feature of patients recovering from periods of intensive care following viral infection, particularly after SARS-CoV-2, is muscle weakness and loss, which may resemble aspects of Guillain-Barre disease, caused by viruses such as Zika, and SARS-CoV-1 and SARS-CoV-2. Ambroxol has been shown to be of have powerful beneficial effects re-establishing neuromuscular function and grip strength by GCase modulation, and other potential mechanisms, in several models on denervation and ALS/motor neurone disease. At least part of this effect will be due to preservation of GM1, a sphingolipid of 5 sugars critical for neuromuscular function and neurotrophins, which is preserved by modulator of GCase, but is also, because of its sialic acid, is a target of cholera toxin, and influenza viruses and cononaviruses. Increased glucosylceramide is the entrance to multiple GSLs, such as GM1 gangliosides, which are able to potentiate neurotrophins and their receptors by interacting with TrkA, facilitating neuronal migration, dendritic arborisation and axonal growth and mitochondrial metabolism. Thus, ambroxol may be administered not only during the early stages of viral infection, but also to help patients who have fatigue and muscle loss to protect from further muscle loss and neuromuscular denervation, but also to recover more quickly. As ambroxol penetrates the brain it may also be used to counter the confusion, memory loss and cognitive dysfunction associated with COVID-19.

Two clinical trials have been initiated by the inventors for ambroxol in India in order to:
1. reduce clinical signs of COVID-19, by reducing the passage of patients infected with SARS-CoV-2 into intensive care, intubation and death in one trial,
2. ameliorate recovery from SARS-CoV-2 infection (Long-COVID) in the second trial.

The inventors have also shown that ambroxol is exceptionally active in reducing dengue infection in host cells. These results are important as ambroxol may be the first direct treatment for dengue virus.

(Kristin et al. 2018) showed that ambroxol had been used in a very few patients with Dengue virus, but only as a symptomatic palliative treatment (table 5, with agents like paracetamol) and not as a specific therapy. To our knowledge there are no references to ambroxol and dengue in scientific literature nor in the patent literature, and not with direct effects on viral replication.

As used herein, the term “prevention” or “prophylaxis” or “preventative treatment” or “prophylactic treatment” comprises a treatment leading to the prevention of a disease as well as a treatment reducing and/or delaying the incidence of a disease or the risk of it occurring. Furthermore the ambroxol will prevent or reverse the effects of the disease by this action.

According to the invention, the modulator of glucosylceramide degradation or a pharmaceutical acceptable salt thereof is particularly useful for preventing or reducing the virus multiplication, and in particular the virus multiplication of SARS-CoV-2. According to the invention, the modulator of glucosylceramide degradation or a pharmaceutical acceptable salt thereof is particularly useful for preventing or reducing muscle loss caused by viral infections, and recovery from intensive care. Advantageously, ambroxol is particularly useful for preventing or reducing the virus multiplication, and in particular the virus multiplication of SARS-CoV-2. Advantageously, ambroxol is particularly useful for preventing or reducing muscle loss caused by viral infections, and recovery from intensive care.

Ambroxol may also be used as a novel treatment for dengue infection, by reducing viral infectivity and reducing clinical effects directly by acting on the infected cells. Thus this is not a symptomatic therapy but the first really targeted therapy for dengue infection, by acting directly on glycosphingolipid metabolism.

As used herein, the term “treatment” or “curative treatment” is defined as a treatment leading to a cure or a treatment which alleviates, improves and/or eliminates, reduces and/or stabilizes the symptoms of a disease or the suffering that it causes.

According to the invention, the modulator of glucosylceramide degradation or a pharmaceutical acceptable salt thereof, as active ingredient, is particularly useful for preventing and/or treating viral infections and disorders associated to the viral infections.

According to the invention, the modulator of glucosylceramide degradation or a pharmaceutical acceptable salt thereof, as a active ingredient, is particularly useful for its use in the treatment and/or the prevention viral infections and disorders associated to the viral infections.

According to the invention, the modulator of glucosylceramide degradation or a pharmaceutical acceptable salt thereof the modulator of glucosylceramide degradation or a pharmaceutical acceptable salt thereof may be used as unique active ingredient.

In an advantageous embodiment, the modulator of glucosylceramide degradation or a pharmaceutical acceptable salt thereof is particularly useful for preventing and/or treating viral infections. In an advantageous embodiment, the modulator of glucosylceramide degradation or a pharmaceutical acceptable salt thereof is particularly useful for preventing and/or treating viral infections involved by enveloped viruses. In particular, viral infections involved by enveloped viruses is selected among the group of Zika infection, dengue infection, influenza infection or coronavirus infection. In an advantageous embodiment, the viral infection is a coronavirus infection. In an advantageous embodiment, the coronavirus infection is selected among the group of human coronavirus 229E, human coronavirus OC43, Severe Acute Respiratory Syndrome coronavirus (SARS-CoV-1), Severe Acute Respiratory Syndrome coronavirus (SARS-CoV-2), human Coronavirus NL63 (HCoV-NL63, New Haven coronavirus), human coronavirus HKU1; and Middle East respiratory syndrome coronavirus (MERS-CoV).

In an advantageous embodiment, the modulator of glucosylceramide degradation or a pharmaceutical acceptable salt thereof is particularly useful for preventing and/or treating disorders associated to the viral infections. In an advantageous embodiment, the disorders associated to the viral infections are selected among the symptoms of Severe Acute Respiratory Syndrome coronavirus (SARS-CoV-2, named “COVID-19”). Advantageously, the disorders associated to the viral infections are selected among the group of cardiac sudden death, cardiac arrhythmias, cardiac infarction, lung damage, muscle loss and weakness including syndromes such as Guillain-Barre syndrome, central nervous system disorders, such as confusion and memory loss, caused by viral infection.

In an advantageous embodiment, ambroxol or a pharmaceutical acceptable salt thereof, as an active ingredient, is particularly useful for preventing and/or treating viral infections and disorders associated to the viral infections. Ambroxol may be used as the unique active ingredient.

Another object of the invention concerns a pharmaceutical composition comprising a modulator of glucosylceramide degradation or a pharmaceutically acceptable salt thereof as active ingredient in an effective amount for treating or preventing viral infections and disorders associated to the viral infections.

Another object of the invention concerns a pharmaceutical composition comprising a modulator of glucosylceramide degradation or a pharmaceutically acceptable salt thereof as active ingredient in an effective amount for its use in the treatment or the prevention of viral infections and disorders associated to the viral infections.

Said modulator of glucosylceramide degradation or a pharmaceutically acceptable salt thereof may be the unique active ingredient in the pharmaceutical composition according to the invention.

Advantageously, the pharmaceutical composition comprising a therapeutically effective amount of the modulator of glucosylceramide degradation or a pharmaceutically acceptable salt thereof as defined above as a active substance, is administered to a subject suffering from or suspected of suffering from viral infections and disorders associated to the viral infections.

By “administered” or “administration” is meant the injection or the delivery to the patient of the pharmaceutical composition according to the invention.

Advantageously, the pharmaceutical composition comprises a therapeutically effective amount of ambroxol or a pharmaceutically acceptable salt thereof as a unique active substance, and is administered to a subject suffering from or suspected of suffering from viral infections and disorders associated to the viral infections.

In one embodiment the subject is a human. In another embodiment the subject is a non-human animal, e.g., a dog, cat, horse, cow, pig, sheep, goat or primate.

Another object of the invention concerns a pharmaceutical composition comprising a pharmaceutically effective amount of the modulator of glucosylceramide degradation or a pharmaceutically acceptable salt thereof as defined above as active substance and at least one pharmaceutically acceptable carrier.

Advantageously, the pharmaceutical composition comprises a pharmaceutically effective amount of ambroxol or a pharmaceutically acceptable salt thereof as active substance and at least one pharmaceutically acceptable carrier.

According to embodiments that involve administering to a subject in need of treatment a therapeutically effective amount of the modulator of glucosylceramide degradation or a pharmaceutically acceptable salt thereof as provided herein, “therapeutically effective” or “an amount effective to treat” or “pharmaceutically effective” denotes the amount of modulator of glucosylceramide degradation or a pharmaceutically acceptable salt thereof or of a composition needed to inhibit or reverse a disease condition (e.g., to treat viral infections and disorders associated to the viral infections). Determining a therapeutically effective amount specifically depends on such factors as toxicity and efficacy of the medicament. These factors will differ depending on other factors such as potency, relative bioavailability, patient body weight, severity of adverse side-effects and preferred mode of administration. Toxicity may be determined using methods well known in the art. Efficacy may be determined utilizing the same guidance. Efficacy, for example, can be measured by a decrease of virus multiplication, and in particular the virus multiplication of SARS-CoV-2. A pharmaceutically effective amount, therefore, is an amount that is deemed by the clinician to be toxicologically tolerable, yet efficacious.

Dosage may be adjusted appropriately to achieve desired drug (e.g., modulator of glucosylceramide degradation or a pharmaceutically acceptable salt thereof or ambroxol) levels, local or systemic, depending upon the mode of administration. In the event that the response in a subject is insufficient at such doses, even higher doses (or effective higher doses by a different, more localized delivery route) may be employed to the extent that patient tolerance permits. Multiple doses per day may also be employed to achieve appropriate systemic levels of the modulator of glucosylceramide degradation or a pharmaceutically acceptable salt thereof. Appropriate systemic levels can be determined by, for example, measurement of the patient's peak or sustained plasma level of the drug. “Dose” and “dosage” are used interchangeably herein.

In an advantageous embodiment, the pharmaceutical composition further comprises a second active ingredient. Non-exhaustive examples of second active ingredient can be selected among the group comprising chloroquine, hydroxychloroquine, ivermectin, ruxolitinib, avdoralimab, remdesivir, interferon beta, azithromycin, lopinavir in combination with ritonavir, lopinavir in combination with ritonavir and interferon beta-1a, antagonists of interleukin 6, such as clazakizumab, tocilizumab and sarilumab, pegylated interferon lambda, doxycycline, favipiravir, nitazoxanide, niclosamide, tranexamic acid, vitamin D, 13 cis retinoic acid, trans retinoic acid, camostat mesilate, immunoglobulins containing anti-Corona VS2 immunoglobulin, açai palm berry extract, ligand of the angiotensin II type 1 receptor (AT1R) such as TRV027, estradiol, undifferentiated allogeneic mesenchymal cells derived from umbilical cord tissue, interleukin 2, Angiotensin Converting Enzyme Inhibitors (ACEI) such as captopril and enalapril, or a combination thereof. Furthermore other drugs which are being developed for COVID-19 listed in https://www.guidetopharmacology.org/GRAC/CoronavirusForward (last update 19 May 2021) may be considered as second active ingredients.

In some embodiments, the compositions provided are employed for in vivo applications. Depending on the intended mode of administration in vivo the compositions used may be in the dosage form of solid, semi-solid or liquid such as, e.g., tablets, pills, powders, capsules, gels, ointments, liquids, suspensions, or the like. Preferably, the compositions are administered in unit dosage forms suitable for single administration of precise dosage amounts. The compositions may also include, depending on the formulation desired, at least one pharmaceutically acceptable carrier or diluent, which are defined as aqueous-based vehicles commonly used to formulate pharmaceutical compositions for animal or human administration. The diluent is selected so as not to affect the biological activity of the modulator of glucosylceramide degradation of interest. Examples of such diluents are distilled water, physiological saline, Ringer's solution, dextrose solution, and Hank's solution. In addition, the pharmaceutical composition may also include other medicinal agents, pharmaceutical agents, carriers, adjuvants, nontoxic, non-therapeutic, non-immunogenic stabilizers, etc. Effective amounts of such diluent or carrier are amounts which are effective to obtain a pharmaceutically acceptable formulation in terms of solubility of components, biological activity, etc. In some embodiments the compositions provided herein are sterile.

The concentration of the modulator of glucosylceramide degradation or a pharmaceutically acceptable salt thereof as defined above in the formulations can vary widely, i.e., from less than 0.5% to 98% by weight, or even more, relative to the total weight of the pharmaceutical composition. In an advantageous embodiments, the concentration of ambroxol or a pharmaceutically acceptable salt thereof as defined above in the formulations can vary widely, i.e., from less than 0.5% to 98% by weight, or even more, relative to the total weight of the pharmaceutical composition.

Administration during in vivo treatment may be by any routes, including oral, parenteral, intramuscular, intranasal, sublingual, intratracheal, inhalation, ocular, vaginal, and rectal. Intracapsular, intravenous, and intraperitoneal routes of administration may also be employed. The skilled artisan recognizes that the route of administration varies depending on the disorder to be treated. For example, the pharmaceutical composition or modulator of glucosylceramide degradation herein may be administered to a subject via oral, parenteral or topical administration. In one embodiment, the compositions or modulator of glucosylceramide degradation herein are administered by oral route, intravenous route, inhalation or nasal spray.

The compositions, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulator agents such as suspending, stabilizing and/or dispersing agents.

Pharmaceutical formulations for parenteral administration include aqueous solutions of the active compositions in water soluble form. Additionally, suspensions of the active compositions may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compositions to allow for the preparation of highly concentrated solutions. Alternatively, the active compositions may be in powder form for constitution with a suitable vehicle, e.g., sterile pyrogen-free water, before use.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. The component or components may be chemically modified so that oral delivery of the modulator of glucosylceramide degradation is efficacious. Generally, the chemical modification contemplated is the attachment of at least one molecule to the modulator of glucosylceramide degradation, where said molecule permits (a) inhibition of proteolysis; and (b) uptake into the blood stream from the stomach or intestine. Also desired is the increase in overall stability of the modulator of glucosylceramide degradation and increase in circulation time in the body. Examples of such molecules include: polyethylene glycol, copolymers of ethylene glycol and propylene glycol, carboxymethyl cellulose, dextran, polyvinyl alcohol, polyvinyl pyrrolidone and polyproline. Other polymers that could be used are poly-1,3-dioxolane and poly-1,3,6-tioxocane. Preferred for pharmaceutical usage, as indicated above, are polyethylene glycol molecules. For oral compositions, the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the modulator of glucosylceramide degradation or by release of the biologically active material beyond the stomach environment, such as in the intestine.

For buccal administration, the compositions may take the form of tablets or lozenges formulated in conventional manner.

For administration by inhalation, the compositions for use according to the present disclosure may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compositions and a suitable powder base such as lactose or starch.

Also contemplated herein is pulmonary delivery. The compositions can be delivered to the lungs of a mammal while inhaling and traverses across the lung epithelial lining to the blood stream. Contemplated for use in the practice of this disclosure are a wide range of mechanical devices designed for pulmonary delivery of therapeutic products, including but not limited to nebulizers, metered dose inhalers, and powder inhalers, all of which are familiar to those skilled in the art.

Nasal delivery of a pharmaceutical composition disclosed herein is also contemplated. Nasal delivery allows the passage of a pharmaceutical composition of the present disclosure to the blood stream directly after administering the therapeutic product to the nose, without the necessity for deposition of the product in the lung. Formulations for nasal delivery include those with dextran or cyclodextran.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compositions, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems.

In a particular advantageous embodiment, the present invention relates to ambroxol or a pharmaceutically acceptable salt thereof as active ingredient in an effective amount for treating or preventing viral infections and disorders associated to the viral infections. Ambroxol or a pharmaceutically acceptable salt thereof may be used as unique active ingredient in an effective amount for treating or preventing viral infections and disorders associated to the viral infections.

In an advantageous embodiments, the pharmaceutical composition comprising the modulator of glucosylceramide degradation, as active ingredient, is administered to the patient at a dose of said modulator of 0.1 to 30 mg/kg of body weight/day. Advantageously, the pharmaceutical composition comprising the modulator of glucosylceramide degradation, as active ingredient, is administered to the patient at a dose of said modulator of 0.1 mg/kg of body weight/day. Advantageously, the pharmaceutical composition comprising the modulator of glucosylceramide degradation, as active ingredient, is administered to the patient at a dose of said modulator of 0.2 mg/kg of body weight/day, advantageously a dose of 0.3 mg/kg of body weight/day, advantageously a dose of 0.4 mg/kg of body weight/day, advantageously a dose of 0.5 mg/kg of body weight/day, advantageously a dose of 0.6 mg/kg of body weight/day, advantageously a dose of 0.7 mg/kg of body weight/day, advantageously a dose of 0.8 mg/kg of body weight/day, advantageously a dose of 0.9 mg/kg of body weight/day, advantageously a dose of 1.0 mg/kg of body weight/day, advantageously a dose of 1.5 mg/kg of body weight/day, advantageously a dose of 2.0 mg/kg of body weight/day, advantageously a dose of 2.5 mg/kg of body weight/day, advantageously a dose of 3.0 mg/kg of body weight/day, advantageously a dose of 3.5 mg/kg of body weight/day, advantageously a dose of 4.0 mg/kg of body weight/day, advantageously a dose of 4.5 mg/kg of body weight/day, advantageously a dose of 5.0 mg/kg of body weight/day, advantageously a dose of 5.5 mg/kg of body weight/day, advantageously a dose of 6.0 mg/kg of body weight/day, advantageously a dose of 6.5 mg/kg of body weight/day, advantageously a dose of 7.0 mg/kg of body weight/day, advantageously a dose of 7.5 mg/kg of body weight/day, advantageously a dose of 8.0 mg/kg of body weight/day, advantageously a dose of 8.5 mg/kg of body weight/day, advantageously a dose of 9.0 mg/kg of body weight/day, advantageously a dose of 9.5 mg/kg of body weight/day, advantageously a dose of 10.0 mg/kg of body weight/day, advantageously a dose of 10.5 mg/kg of body weight/day, advantageously a dose of 11.0 mg/kg of body weight/day, advantageously a dose of 11.5 mg/kg of body weight/day, advantageously a dose of 12.0 mg/kg of body weight/day, advantageously a dose of 12.5 mg/kg of body weight/day, advantageously a dose of 13.0 mg/kg of body weight/day, advantageously a dose of 13.5 mg/kg of body weight/day, advantageously a dose of 14.0 mg/kg of body weight/day, advantageously a dose of 14.5 mg/kg of body weight/day, advantageously a dose of 15.0 mg/kg of body weight/day, advantageously a dose of 15.5 mg/kg of body weight/day, advantageously a dose of 16.0 mg/kg of body weight/day, advantageously a dose of 16.5 mg/kg of body weight/day, advantageously a dose of 17.0 mg/kg of body weight/day, advantageously a dose of 17.5 mg/kg of body weight/day, advantageously a dose of 18.0 mg/kg of body weight/day, advantageously a dose of 18.5 mg/kg of body weight/day, advantageously a dose of 19.0 mg/kg of body weight/day, advantageously a dose of 19.5 mg/kg of body weight/day, advantageously a dose of 20.0 mg/kg of body weight/day, advantageously a dose of 20.5 mg/kg of body weight/day, advantageously a dose of 21.0 mg/kg of body weight/day, advantageously a dose of 21.5 mg/kg of body weight/day, advantageously a dose of 22.0 mg/kg of body weight/day, advantageously a dose of 22.5 mg/kg of body weight/day, advantageously a dose of 23.0 mg/kg of body weight/day, advantageously a dose of 23.5 mg/kg of body weight/day, advantageously a dose of 24.0 mg/kg of body weight/day, advantageously a dose of 24.5 mg/kg of body weight/day, advantageously a dose of 25.0 mg/kg of body weight/day, advantageously a dose of 25.5 mg/kg of body weight/day, advantageously a dose of 26.0 mg/kg of body weight/day, advantageously a dose of 26.5 mg/kg of body weight/day, advantageously a dose of 27.0 mg/kg of body weight/day, advantageously a dose of 27.5 mg/kg of body weight/day, advantageously a dose of 28.0 mg/kg of body weight/day, advantageously a dose of 28.5 mg/kg of body weight/day, advantageously a dose of 29.0 mg/kg of body weight/day, advantageously a dose of 29.5 mg/kg of body weight/day, advantageously a dose of 30.0 mg/kg of body weight/day. In an advantageous embodiments, the pharmaceutical composition comprising the modulator of glucosylceramide degradation, as active ingredient, is administered to the patient at a dose of said modulator of 0.3 to 15 mg/kg of body weight/day.

In an advantageous embodiment, the pharmaceutical composition comprising ambroxol, as active ingredient, is administered to the patient at a dose of said modulator of 0.1 to 30 mg/kg of body weight/day. In an advantageous embodiments, the pharmaceutical composition comprising ambroxol, as unique active ingredient, is administered to the patient at a dose of said modulator of 0.3 to 15 mg/kg of body weight/day.

In other embodiments, the pharmaceutical composition comprising the modulator of glucosylceramide degradation, as active ingredient, is administered to the patient at a dose of 0 said modulator of 0.1 to 30 mg/kg of body weight/day with a target plasma level of 10 to 1000 ng/ml. In other embodiments, the pharmaceutical composition comprising the modulator of glucosylceramide degradation, as active ingredient, is administered to the patient at a dose of said modulator of 0.3 to 15 mg/kg of body weight/day, with a target plasma level of 10 to 1000 ng/ml.

In other embodiments, the pharmaceutical composition comprising ambroxol, as unique active ingredient, is administered to the patient at a dose of 0.1 to 30 mg/kg of body weight/day with a target plasma level of 10 to 1000 ng/ml. In other embodiments, the pharmaceutical composition comprising ambroxol, as unique active ingredient, is administered to the patient at a dose of 0.3 to 15 mg/kg of body weight/day, with a target plasma level of 10 to 1000 ng/ml.

Another subject of the invention relates to a method for the preventive treatment of viral infections and disorders associated to the viral infections in patients in need of, comprising the administration to said patients of a pharmaceutical composition comprising a therapeutically effective amount of a modulator of glucosylceramide degradation or a pharmaceutically acceptable salt thereof as an active substance and at least one pharmaceutically acceptable carrier as defined above. Advantageously, the viral infections are viral infections involved by enveloped viruses. In particular, viral infections involved by enveloped viruses is selected among the group of Zika infection, Dengue infection, influenza infection or coronavirus infection. Advantageously, the coronavirus infection is selected among the group of human coronavirus 229E, human coronavirus OC43, Severe Acute Respiratory Syndrome coronavirus (SARS-CoV-1), Severe Acute Respiratory Syndrome coronavirus (SARS-CoV-2), human Coronavirus NL63 (HCoV-NL63, New Haven coronavirus), human coronavirus HKU1; and Middle East respiratory syndrome coronavirus (MERS-CoV). Advantageously, the disorders associated to the viral infections are selected among the group of cardiac sudden death, cardiac arrhythmias, cardiac infarction, lung damage, muscle loss and weakness including syndromes such as Guillain-Barre syndrome, central nervous system disorders, such as confusion and memory loss, caused by viral infection.

In a particular advantageous embodiment, the present invention relates to the use of a pharmaceutical composition comprising a therapeutically effective amount of a modulator of glucosylceramide degradation or a pharmaceutically acceptable salt thereof as an active in the manufacture of a medicinal product intended for the prevention of viral infections and disorders associated to the viral infections.

In a particular advantageous embodiment, the present invention relates to the use of a pharmaceutical composition comprising a therapeutically effective amount of a modulator of glucosylceramide degradation or a pharmaceutically acceptable salt thereof as an active in the manufacture of a medicinal product intended for the prevention of viral infections and disorders associated to the viral infections.

Another subject of the invention relates to a method for the preventive treatment of viral infections and disorders associated to the viral infections in patients in need of, comprising the administration to said patients of a pharmaceutical composition comprising a therapeutically effective amount of ambroxol or a pharmaceutically acceptable salt thereof as an active substance and at least one pharmaceutically acceptable carrier as defined above. Advantageously, the viral infections are viral infections involved by enveloped viruses. In particular, viral infections involved by enveloped viruses is selected among the group of Zika infection, dengue infection, influenza infection or coronavirus infection. Advantageously, the coronavirus infection is selected among the group of human coronavirus 229E, human coronavirus OC43, Severe Acute Respiratory Syndrome coronavirus (SARS-CoV-1), Severe Acute Respiratory Syndrome coronavirus (SARS-CoV-2), human Coronavirus NL63 (HCoV-NL63, New Haven coronavirus), human coronavirus HKU1; and Middle East respiratory syndrome coronavirus (MERS-CoV). Advantageously, the disorders associated to the viral infections are selected among the group of cardiac sudden death, cardiac arrhythmias, cardiac infarction, lung damage, muscle loss and weakness including syndromes such as Guillain-Barre syndrome, central nervous system disorders, such as confusion and memory loss, caused by viral infection.

In a particular advantageous embodiment, the present invention relates to the use of a pharmaceutical composition comprising a therapeutically effective amount of ambroxol as an active in the manufacture of a medicinal product intended for the prevention of viral infections and disorders associated to the viral infections.

In a particular advantageous embodiment, the present invention relates to the use of a pharmaceutical composition comprising a therapeutically effective amount of ambroxol as an active in the manufacture of a medicinal product intended for the prevention of viral infections and disorders associated to the viral infections.

Another subject of the invention relates to a method for the curative treatment of viral infections and disorders associated to the viral infections in patients in need of, comprising the administration to said patients of a pharmaceutical composition comprising a therapeutically effective amount of a modulator of glucosylceramide degradation or a pharmaceutically acceptable salt thereof as an active substance and at least one pharmaceutically acceptable carrier as defined above. Advantageously, the viral infections are viral infections involved by enveloped viruses. In particular, viral infections involved by enveloped viruses is selected among the group of Zika infection, dengue infection, influenza infection or coronavirus infection. Advantageously, the coronavirus infection is selected among the group of human coronavirus 229E, human coronavirus OC43, Severe Acute Respiratory Syndrome coronavirus (SARS-CoV-1), Severe Acute Respiratory Syndrome coronavirus (SARS-CoV-2), human Coronavirus NL63 (HCoV-NL63, New Haven coronavirus), human coronavirus HKU1; and Middle East respiratory syndrome coronavirus (MERS-CoV). Advantageously, the disorders associated to the viral infections are selected among the group of cardiac sudden death, cardiac arrhythmias, cardiac infarction, lung damage, muscle loss and weakness including syndromes such as Guillain-Barre syndrome, central nervous system disorders, such as confusion and memory loss, caused by viral infection.

In a particular advantageous embodiment, the present invention relates to the use of a pharmaceutical composition comprising a therapeutically effective amount of a modulator of glucosylceramide degradation or a pharmaceutically acceptable salt thereof as an active in the manufacture of a medicinal product intended for the curative treatment of viral infections and disorders associated to the viral infections.

In a particular advantageous embodiment, the present invention relates to the use of a pharmaceutical composition comprising a therapeutically effective amount of a modulator of glucosylceramide degradation or a pharmaceutically acceptable salt thereof as an active in the manufacture of a medicinal product intended for the curative treatment of viral infections and disorders associated to the viral infections.

Another subject of the invention relates to a method for the curative treatment of viral infections and disorders associated to the viral infections in patients in need of, comprising the administration to said patients of a pharmaceutical composition comprising a therapeutically effective amount of ambroxol or a pharmaceutically acceptable salt thereof as an active substance and at least one pharmaceutically acceptable carrier as defined above. Advantageously, the viral infections are viral infections involved by enveloped viruses. In particular, viral infections involved by enveloped viruses is selected among the group of Zika infection, Dengue infection, influenza infection or coronavirus infection. Advantageously, the coronavirus infection is selected among the group of human coronavirus 229E, human coronavirus OC43, Severe Acute Respiratory Syndrome coronavirus (SARS-CoV-1), Severe Acute Respiratory Syndrome coronavirus (SARS-CoV-2), human Coronavirus NL63 (HCoV-NL63, New Haven coronavirus), human coronavirus HKU1; and Middle East respiratory syndrome coronavirus (MERS-CoV).

Advantageously, the disorders associated to the viral infections are selected among the group of cardiac sudden death, cardiac arrhythmias, cardiac infarction, lung damage, muscle loss and weakness including syndromes such as Guillain-Barre syndrome, central nervous system disorders, such as confusion and memory loss, caused by viral infection.

In a particular advantageous embodiment, the present invention relates to the use of a pharmaceutical composition comprising a therapeutically effective amount of ambroxol as an active in the manufacture of a medicinal product intended for the curative treatment of viral infections and disorders associated to the viral infections.

In a particular advantageous embodiment, the present invention relates to the use of a pharmaceutical composition comprising a therapeutically effective amount of ambroxol as an active in the manufacture of a medicinal product intended for the curative treatment of viral infections and disorders associated to the viral infections.

Another subject of the invention relates to a method of treating or preventing viral infections and disorders associated to the viral infections, comprising administering to the patient the pharmaceutical composition comprising a therapeutically effective amount of ambroxol or a pharmaceutically acceptable salt thereof as an active substance and at least one pharmaceutically acceptable carrier as defined above. In an advantageously embodiment, the composition further comprises at least one pharmaceutically acceptable carrier. In an advantageously embodiment, the composition further comprises a second active ingredient. In an advantageously embodiment, the composition is administered by oral route, intravenous route, inhalation or nasal spray. Advantageously, the viral infections are viral infections involved by enveloped viruses. In particular, viral infections involved by enveloped viruses is selected among the group of Zika infection, dengue infection, influenza infection or coronavirus infection. Advantageously, the coronavirus infection is selected among the group of human coronavirus 229E, human coronavirus OC43, Severe Acute Respiratory Syndrome coronavirus (SARS-CoV-1), Severe Acute Respiratory Syndrome coronavirus (SARS-CoV-2), human Coronavirus NL63 (HCoV-NL63, New Haven coronavirus), human coronavirus HKU1; and Middle East respiratory syndrome coronavirus (MERS-CoV). Advantageously, the disorders associated to the viral infections are selected among the group of cardiac sudden death, cardiac arrhythmias, cardiac infarction, lung damage, muscle loss and weakness including syndromes such as Guillain-Barre syndrome, central nervous system disorders, such as confusion and memory loss, caused by viral infection.

The invention is further illustrated by the following examples in connection with FIGS. 1 and 2.

### EXAMPLES

### Example 1: Antiviral Activity of Ambroxol Against SARS-CoV-2 in A549-ACE2 Cells

1.1. Material and Method

Cells were seeded in 96-well plates at 2.10⁴ cells per well at day 0 in DMEM 10% FBS, 1% P/S. The next day, cells were pretreated for 2 hours prior to infection with 5 concentrations of the ambroxol in triplicate (10⁻⁸ to 5.10⁻⁵ M) or as a control, with the equivalent volume of maximum vehicle used (DMSO) in DMEM 2% FBS, 1% P/S, and moved to the BSL-3. The media was set aside, and replaced with an inoculum of SARS-CoV-2 (ratio 1 for A549-ACE2 cells) in DMEM 0% FBS, with different concentration of ambroxol, and incubated at 37° C. for 2 hours. The inoculum was then removed, and replaced with 100 μL of 2% FBS media containing the different concentrations of ambroxol. The plates were taped, and incubated at 37° C. for 48 h. At 48 h post infection, supernatants were harvested, centrifugated 5 mn at 450 g, and transferred to a fresh tube. RNA was extracted using the QIAamp viral RNA extraction kit following the manufacturer instructions. Measurement of viral replication was performed by quantitative RT-PCR in duplicates in a one-step reaction using the NEB Universal Probe One-Step RT-qPCR Kit. In parallel, cytotoxicity was assessed on a replicate plate (seeded the same day as for the infection) after 48 h incubation with the different concentrations of ambroxol, using the AlamarBlue kit (Invitrogen).
1.2 Results:

Results are presented in FIGS. 1A, 1B and 1C.

Ambroxol shows a significant concentration-dependent antiviral effect on SARS-CoV-2 replication (dot line “ [symbol] ” in FIG. 1A and 1B) with no toxic effects on cell viability (square lines “ [symbol] ”) in the A549-ACE2 cells. FIG. 1B shows the same data with viral load reported as a ratio of the no treatment condition (DMSO) with a clear concentration-dependent effect.

This effect is compatible with an effect on the host A549 cells rather than a direct antiviral effect on viral replication, but indicates that ambroxol may influence favorably infection at the same concentrations as the drug modifies glucosylceramidase and neuromuscular function.

The antiviral effect of Ambroxol is dose-dependent as shown by the dose-response curve on FIG. 1C. The 50% effective concentration (IC50) is 22 μM. In these experiments the maximal antiviral effect was equivalent to that of remdesivir.

### Example 2: Critical role of glucosylceramide and glucosylceramidase (GBA1 and GBA2) in viral infection and antiviral activity of Ambroxol against Dengue virus in Huh7 cells.

2.1 Material and methods Huh7, human hepatoma cells were used for all primary experiments. For all siRNA experiments, cells were seeded in 24-well plates at a concentration of 2×10⁵ cells/well in DMEM and 10% FBS. For all ambroxol experiments, cells were seeded in 96 well plates at a concentration of 2×10⁵ cells/well in Dulbecco's Modified Eagle Medium (DMEM) and 10% fetal bovine serum (FBS). During siRNA and drug treatment the media was removed and replaced with DMEM and 2% FBS and a corresponding concentration of siRNA or ambroxol. During viral infection, all media is removed and set aside, and a viral inoculum is added to the cells. The cells and viral inoculum are incubated for 1 hr at room temperature on a rocker. The virus is then aspirated from cells, cells are washed with 1Xphosphate buffer saline (PBS), the media is re-added to the cells, and cells are incubated at 37° C. and 5% CO₂ for 24 h. In parallel, cytotoxicity of ambroxol and/or siRNA was assessed on a replicate plate (seeded the same day as for the infection) treated with the same concentrations of siRNA and/or ambroxol as the infection utilizing an AlamarBlue assay (Invitrogen). Plaque assay analysis of infectious virus release from supernatants was conducted on baby hamster kidney cells (BHK cells). BHK cells are maintained in MEM with 10% FBS. For plaque assays, BHKs are seeded in 6 well plates at a density of 6×10⁶ cells/well. The following day, dilutions of viral supernatants collected from samples are applied to BHK cells, followed by a 1 hr incubation at room temperature, and then cells are overlaid with MEM containing 5% FBS and 2% agarose. Plaques are counted at 6 days post infection. For qRT-PCR analysis, cells are collected post infection in TRIzol reagent (Invitrogen), and RNA is extracted per manufacturer's protocol. A one-step qRT-PCR kit from Agilent (Brilliant III Ultra Fast SYBR Green qRT-PCR Kit) is used to assess viral genome copies. A standard curve of viral RNA is generated using a viral RNA from a cDNA subclone of DENV2 as described in Gullberg RC, et al. 2018. Copies of viral RNA are normalized to the ribosomal protein lateral stalk protein subunit PO (RPLPO) gene using the delta delta Cq method also described in Gullberg RC et al. 2018 (cited above).

Huh7 cells were treated with siRNA targeting glucosylceramidase beta 1 (GBA1) and infected with dengue virus serotype 2—strain 16681 (DENV2) with a multiplicity of infection or MOI=0.3) according to known techniques (Gullberg RC et al. 2018 cited above). After 24 hours post infection (hpi), supernatants were collected and analyzed via plaque assay on baby hamster kidney cells (BHK cells) for infectious virus release. Results are given in FIG. 2A.

Huh7 cells were pre-treated with Ambroxol hydrochloride at 0.391, 0.781, 1.56, 3.125, 6.25, 12.5, 25, 50, 100, and 200 μM for 24 h, followed by DENV2 infection with MOI=0.1; or MOI=5). 24 hours post infection, supernatants were collected and analyzed via plaque assay on BHK cells. Results are given in FIG. 2B and 2C.

Huh7 cells were treated with siRNAs targeting GBA1 and glucosylceramidase beta 2 (GBA2) enzymes followed by DENV2 infection with MOI=0.1; or MOI=5). 24 hpi, supernatants were collected and analyzed via plaque assay on BHK cells while RNA was extracted from cells and analyzed via qRT-PCR for DENV2 genome replication. Results are given in FIG. 2D-G
2.2. Results

They are given in FIG. 2A-G.

In the loss of function study, it was found that siRNA-mediated inhibition of the lysosomal glucosylceramidase beta (GBA1) gene, resulted in an increase of infectious dengue virus serotype 2 (DENV2) from human hepatoma cells (Huh7 cells) (FIG. 2A). This implicated a potential antiviral role for GBA1.

In order to demonstrate that the inhibition of the non-lysosomal glucosylceramidase beta (GBA2) would have a similar effect, Ambroxol hydrocholoride, a known GBA1 chaperone and GBA2 inhibitor, have been tested on DENV2-infected Huh7 cells against both a high and low multiplicity of infection (MOI). In low MOI infection (MOI=0.1, FIG. 2B), all concentrations of Ambroxol HCl tested resulted in a statistically significant reduction of DENV2 release from Huh7 cells. At concentrations above 6.25 μM, there was no detectable virus released from cells, with only the highest concentrations (100 μM and 200 μM) having any cytopathic effect on cells. In a high MOI infection (MOI=5, FIG. 2C), was also showed that all concentrations of Ambroxol HCl reduced DENV2 release; however, only concentrations of 12.5 μM and above were statistically significant.

To further validate that the inhibitory effect of Ambroxol against DENV2 was due to its effect on GBA1 and GBA2, an additional siRNA-mediated loss of function study of GBA1 and GBA2 at both high and low MOI have been performed (FIG. 2D-G). A loss of GBA1 function resulted in an increase of DENV2 release and viral genome replication while loss of GBA2 function resulted in a decrease in viral release and replication at low MOI compared to the untreated control (FIG. 2D, E). At high MOI, the loss of GBA1 function no longer showed an increase in DENV2 release compared to the untreated control, despite a clear increase in viral replication (FIG. 2F, G). Loss of function of GBA2 at high MOI, however, still showed a significant decrease in viral release at high MOI.

Taken together, these data indicate that spatial control of glucosylceramide hydrolysis by GBA1 and GBA2 is a critical node within the DENV2 lifecycle. Specifically, an increase in GBA1 activity while suppression of GBA2 activity appears to have potent antiviral potential which uniquely positions Ambroxol hydrochloride as a promising therapeutic agent against dengue viruses. Of additional interest, these experiments also showed that ambroxol hydrochloride is an effective inhibitor of dengue virus when applied at both high and low concentrations. Lower concentrations of virus are more reflective of biological conditions when patients are infected with dengue. It is striking that our data also shows ambroxol hydrochloride was able to inhibit dengue at higher concentrations as high concentrations of virus are generally able to overcome inhibitory effects of many compounds.

### REFERENCES

Bouscary, Alexandra, Cyril Quessada, Althéa Mosbach, Noëlle Callizot, Michael Spedding, Jean-Philippe Loeffler, and Alexandre Henriques. 2019. ‘Ambroxol Hydrochloride Improves Motor Functions and Extends Survival in a Mouse Model of Familial Amyotrophic Lateral Sclerosis’. Frontiers in Pharmacology 10: 883. https://doi.org/10.3389/fphar.2019.00883.
Bouscary, Alexandra, Cyril Quessada, Frédérique René, Michael Spedding, Bradley J. Turner, Alexandre Henriques, Shyuan T. Ngo, and Jean-Philippe Loeffler. 2020. ‘Sphingolipids Metabolism Alteration in the Central Nervous System: Amyotrophic Lateral Sclerosis (ALS) and Other Neurodegenerative Diseases’. Seminars in Cell & Developmental Biology, November. https://doi.org/10.1016/j.semcdb.2020.10.008.
Dodge, James C., Christopher M. Treleaven, Joshua Pacheco, Samantha Cooper, Channa Bao, Marissa Abraham, Mandy Cromwell, et al. 2015. ‘Glycosphingolipids Are Modulators of Disease Pathogenesis in Amyotrophic Lateral Sclerosis’. Proceedings of the National Academy of Sciences of the United States of America 112 (26): 8100-8105. https://doi.org/10.1073/pnas.1508767112.
Gullberg Rebekah C, J Jordan Steel, Venugopal Pujari, Joel Rovnak, Dean C Crick, Rushika Perera. 2018. ‘Stearoyl-CoA desaturase 1 differentiates early and advanced dengue virus infections and determines virus particle infectivity.’ PLoS Pathogens 14(8):E1007261.
Henriques, Alexandre, Vincent Croixmarie, Alexandra Bouscary, Althéa Mosbach, Céline Keime, Claire Boursier-Neyret, Bernard Walter, Michael Spedding, and Jean-Philippe Loeffler. 2017. ‘Sphingolipid Metabolism Is Dysregulated at Transcriptomic and Metabolic Levels in the Spinal Cord of an Animal Model of Amyotrophic Lateral Sclerosis’. Frontiers in Molecular Neuroscience 10: 433. https://doi.org/10.3389/fnmol.2017.00433.
Henriques, Alexandre, Vincent Croixmarie, David A. Priestman, Angela Rosenbohm, Sylvie Dirrig-Grosch, Eleonora D'Ambra, Mylene Huebecker, et al. 2015. ‘Amyotrophic Lateral Sclerosis and Denervation Alter Sphingolipids and Up-Regulate Glucosylceramide Synthase’. Human Molecular Genetics 24 (25): 7390-7405. https://doi.org/10.1093/hmeddv439.
Henriques, Alexandre, Mylene Huebecker, Hélène Blasco, Céline Keime, Christian R. Andres, Philippe Corcia, David A. Priestman, Frances M. Platt, Michael Spedding, and Jean-Philippe Loeffler. 2017. ‘Inhibition of β-Glucocerebrosidase Activity Preserves Motor Unit Integrity in a Mouse Model of Amyotrophic Lateral Sclerosis’. Scientific Reports 7 (1): 5235. https://doi.org/10.1038/s41598-017-05313-0.
Kristin, Erna, Rizaldy Taslim Pinzon, Woro Rukmi Pratiwi, Christiana Trijayanti, Ery Setiawan, VettyYulianti Permanasari, and Mardiati Nadjib. 2018. ‘Drug Use Pattern in Dengue Patients Who Visited Hospitals and Primary Health Center in Yogyakarta, Indonesia’. J. Pharm. Sci. 10: 4.
Vitner, Einat B., Hagit Achdout, Roy Avraham, Boaz Politi, Lilach Cherry, Hadas Tamir, Yfat Yahalom-Ronen, et al. 2021. ‘Glucosylceramide Synthase Inhibitors Prevent Replication of SARS-CoV-2 and Influenza Virus’. The Journal of Biological Chemistry, February, 100470. https://doi.org/10.1016/j.jbc.2021.100470.
Ziegler, Carly G. K., Samuel J. Allon, Sarah K. Nyquist, Ian M. Mbano, Vincent N. Miao, Constantine N. Tzouanas, Yuming Cao, et al. 2020. ‘SARS-CoV-2 Receptor ACE2 Is an Interferon-Stimulated Gene in Human Airway Epithelial Cells and Is Detected in Specific Cell Subsets across Tissues’. Cell 181 (5): 1016-1035.e19. https://doi.org/10.1016/j.cell.2020.04.035.

1-23. (canceled)

24. A modulator of glucosylceramide degradation or a pharmaceutical acceptable salt thereof, as active ingredient, in an effective amount for treating or preventing viral infections and disorders associated to the viral infections.

25. A modulator of glucosylceramide degradation or a pharmaceutical acceptable salt thereof, as a active ingredient, in an effective amount for its use for the treatment or the prevention of viral infections and disorders associated to the viral infections.

26. The modulator of glucosylceramide degradation or a pharmaceutical acceptable salt thereof as defined in claim 24, which is used as unique active ingredient.

27. The modulator of glucosylceramide degradation or a pharmaceutical acceptable salt thereof as defined in claim 24, wherein the modulator of glucosylceramide degradation or a pharmaceutical acceptable salt thereof binds to beta-glucocerebrosidase 1 (GBA1) or the beta-glucocerebrosidase 2 (GBA2) or both and may inhibit or may act as an enzyme chaperone.

28. The modulator of glucosylceramide degradation or a pharmaceutical acceptable salt thereof as defined in claim 24, wherein the modulator of glucosylceramide degradation is ambroxol or a pharmaceutically acceptable salt thereof.

29. The modulator of glucosylceramide degradation as defined in claim 24, wherein the viral infections are selected among the group of Zika infection, dengue infection, influenza infection and coronavirus infection.

30. The modulator of glucosylceramide degradation according to claim 29, wherein the coronavirus infection is selected among the group of human coronavirus 229E, human coronavirus OC43, Severe Acute Respiratory Syndrome coronavirus (SARS-CoV-1), Severe Acute Respiratory Syndrome coronavirus (SARS-CoV-2), human Coronavirus NL63 (HCoV-NL63, New Haven coronavirus), human coronavirus HKU1; and Middle East respiratory syndrome coronavirus (MERS-CoV).

31. The modulator of glucosylceramide degradation as defined in claim 24, wherein the disorders associated to the viral infections are selected among the group of cardiac sudden death, cardiac arrhythmias, cardiac infarction, lung damage, muscle loss and weakness including syndromes such as Guillain-Barre syndrome, central nervous system disorders, such as confusion and memory loss, caused by viral infection.

32. A pharmaceutical composition comprising a modulator of glucosylceramide degradation or a pharmaceutically acceptable salt thereof as defined in claim 24 as active ingredient in an effective amount for treating or preventing viral infections and disorders associated to the viral infections.

33. A pharmaceutical composition comprising a modulator of glucosylceramide degradation or a pharmaceutically acceptable salt thereof as defined in claim 25 as active ingredient in an effective amount for its use for the treatment or the prevention of viral infections and disorders associated to the viral infections.

34. A pharmaceutical composition comprising a modulator of glucosylceramide degradation or a pharmaceutically acceptable salt thereof as defined in claim 24, wherein said modulator is a unique active ingredient.

35. The pharmaceutical composition as defined in claim 32, further comprising at least one pharmaceutically acceptable carrier.

36. The pharmaceutical composition as defined in claim 32, further comprising a second active ingredient.

37. The pharmaceutical composition as defined in claim 32, wherein the composition is administered by oral route, intravenous route, inhalation or nasal spray.

38. The pharmaceutical composition as defined in claim 32, wherein the pharmaceutical composition is administered to the patient at a dose of said modulator of 0.1 to 30 mg/kg of body weight/day.

39. The pharmaceutical composition as defined in claim 38, wherein the pharmaceutical composition is administered to the patient at a dose of said modulator of 0.3 to 15 mg/kg of body weight/day, with a target plasma level of 10-1000 ng/ml.

40. The pharmaceutical composition as defined in claim 32, wherein the viral infections are selected among the group of Zika infection, dengue infection, influenza infection and coronavirus infection.

41. The pharmaceutical composition as defined in claim 40, wherein the coronavirus infection is selected among the group of human coronavirus 229E, human coronavirus OC43, Severe Acute Respiratory Syndrome coronavirus (SARS-CoV-1), severe Acute Respiratory Syndrome coronavirus (SARS-CoV-2), human coronavirus NL63 (HCoV-NL63, New Haven coronavirus), human coronavirus HKU1, and Middle East respiratory syndrome coronavirus (MERS-CoV).

42. The pharmaceutical composition as defined in claim 32, wherein the disorders associated to the viral infections are selected among the group of cardiac sudden death, cardiac arrhythmias, cardiac infarction, lung damage, muscle loss and weakness including syndromes such as Guillain-Barre syndrome, central nervous system disorders, such as confusion and memory loss, caused by viral infection.

43. A method of treating or preventing viral infections and disorders associated to the viral infections, comprising administering to the patient the pharmaceutical composition as defined in claim 32.

44. The method according to claim 43, wherein the composition further comprises at least one pharmaceutically acceptable carrier.

45. The method according to claim 43, wherein the composition further comprises a second active ingredient.

46. The method according to claim 43, wherein the composition is administered by oral route, intravenous route, inhalation or nasal spray.
